(19) United States
(12) Patent Application Publication
(10) Pub. No.: US 2025/0295352 A1
(43) Pub. Date: Sep. 25, 2025

(54) AUTOMATED REPORT GENERATION FOR AUTISM SPECTRUM DISORDER (ASD)

(71) Applicant: mHealthcare, Inc., New York, NY (US)

(72) Inventors: Shamshad Ansari, Centerville, VA (US); Michael E. Johnson, Las Vegas, NV (US)

(21) Appl. No.: 19/084,589

(22) Filed: Mar. 19, 2025

Related U.S. Application Data
(60) Provisional application No. 63/567,129, filed on Mar. 19, 2024.

(51) Int. Cl. A61B 5/00; G06T 7/00; G06V 10/70; G06V 10/77; G06V 10/82; G06V 20/40; G06V 40/20; G10L 15/16; G10L 15/183; G10L 15/22; G10L 25/57; G10L 25/66; G16H 15/00; G16H 50/20

(52) U.S. Cl. CPC A61B 5/4076; A61B 5/7264; G06T 7/0012; G06V 10/7715; G06V 10/82; G06V 20/46; G06V 40/20; G10L 15/16; G10L 15/183; G10L 15/22; G10L 25/57; G10L 25/66; G16H 15/00; G16H 50/20; G06T 2207/10016; G06T 2207/20081; G06T 2207/20084; G06T 2207/30004; G06T 2207/30196; G06V 10/768; G06V 2201/03; G10L 2015/223

(57) ABSTRACT

Systems and methods that generate reports for assessment sessions are described. For example, an assessment system may automatically process audiovisual data (e.g., a voice command synced to captured video of an assessment session) in real-time, extract relevant features, and generate an assessment report or perform other actions. The systems and methods, therefore, may facilitate an efficient and accurate generation of diagnostic reports for an assessment session (e.g., for ASD), enabling remote diagnosis while incorporating human oversight for final approval, among other benefits.

### CROSS-REFERENCE TO RELATED APPLICATIONS

This application claims priority to U.S. Provisional Patent Application No. 63/567,129, filed on Mar. 19, 2024, entitled REPORT GENERATING SYSTEM FOR THE DIAGNOSIS OF AUTISM SPECTRUM DISORDER (ASD) USING MULTIMODAL ANALYSIS AND ARTIFICIAL INTELLIGENCE, which is hereby incorporated by reference in its entirety.

### BACKGROUND

Autism Spectrum Disorder (ASD) is a developmental condition that may present challenges to people, often children, such as challenges associated with social interactions and communication, repetitive activities, narrow or restricted interests or activities, or other similar behaviors. Traditionally, a diagnosis of ASD is based on clinical observations, structured behavioral assessments, digital screening, and standardized questionnaires. Such methods may be time-consuming, resource-intensive, and/or limited by geographical access to specialists or by a limited information base of a subject and observed behaviors.

### BRIEF DESCRIPTION OF THE DRAWINGS

FIG. 1 is a block diagram illustrating a suitable network environment for generating reports associated with an ASD assessment of a patient.

FIG. 2 is a block diagram illustrating an assessment system.

FIG. 3 is a flow diagram illustrating a method of generating a diagnostic report for an assessment session.

FIG. 4 is a flow diagram illustrating a method of generating command-action pairs from audiovisual data.

FIG. 5 is a flow diagram illustrating the generation of a report.

FIG. 6 is a flow diagram illustrating a method for generating a report associated with an assessment session.

In the drawings, some components are not drawn to scale, and some components and/or operations can be separated into different blocks or combined into a single block for discussion of some of the implementations of the present technology. Moreover, while the technology is amenable to various modifications and alternative forms, specific implementations have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

### Overview

The technology described herein relates to an automated telehealth assessment report generation system for diagnosing ASD in humans, such as children. The systems and methods, in some embodiments, utilize a combination of machine learning (ML), computer vision, and/or generative artificial intelligence (AI) to analyze multi-modal inputs and perform actions, such as determining/inferring impressions for a subject. The inputs may include video feeds, voice feeds, sensory-motor biometrics, and/or expert commands during remote assessments conducted via teleconferencing platforms, which may be de-identified, HIPAA-compliant and/or time-synced.

As described herein, existing telehealth solutions lack automated, AI-driven, and real-time diagnostic capabilities. The systems and methods described herein enable a robust, automated telehealth system that aligns voice commands with video analysis to enhance diagnostic precision and accessibility. For example, a licensed Ph.D. psychologist, developmental Pediatrician, or other expert evaluator interacts with a child through voice commands. The parents of the child assist the child in executing the commands and hold a camera to the child, capturing video of the child's reactions, behaviors, and/or movements to the commands.

The systems and methods may automatically process the audiovisual data (e.g., the voice command synced to the captured video) in real-time, extract relevant features, and generate an assessment report or perform other actions. The systems and methods, therefore, may facilitate an efficient and accurate generation of diagnostic reports for ASD, enabling remote diagnosis while incorporating human oversight for final approval, among other benefits.

In the following description, for the purposes of explanation, numerous specific details are set forth in order to provide a thorough understanding of implementations of the present technology. It will be apparent, however, to one skilled in the art that implementations of the present technology can be practiced without some of these specific details. The phrases “in some implementations,” “according to some implementations,” “in the implementations shown,” “in other implementations,” and the like generally mean the particular feature, structure, or characteristic following the phrase is included in at least one implementation of the present technology and can be included in more than one implementation. In addition, such phrases do not necessarily refer to the same implementations or different implementations.

### Examples of an ASD Assessment System

As described herein, in some embodiments, an ASD assessment system, or assessment system, incorporates and/or provides a telehealth-based automated ASD diagnostic report generator that integrates multi-modal AI processing, real-time data synchronization, and explainable AI (XAI) tools. The system analyzes synchronized video, audio, and natural language inputs, generates structured reports, and facilitates clinician oversight (e.g., for final validation), enhances the accuracy and efficiency of an ASD diagnosis of a subject, such as a child.

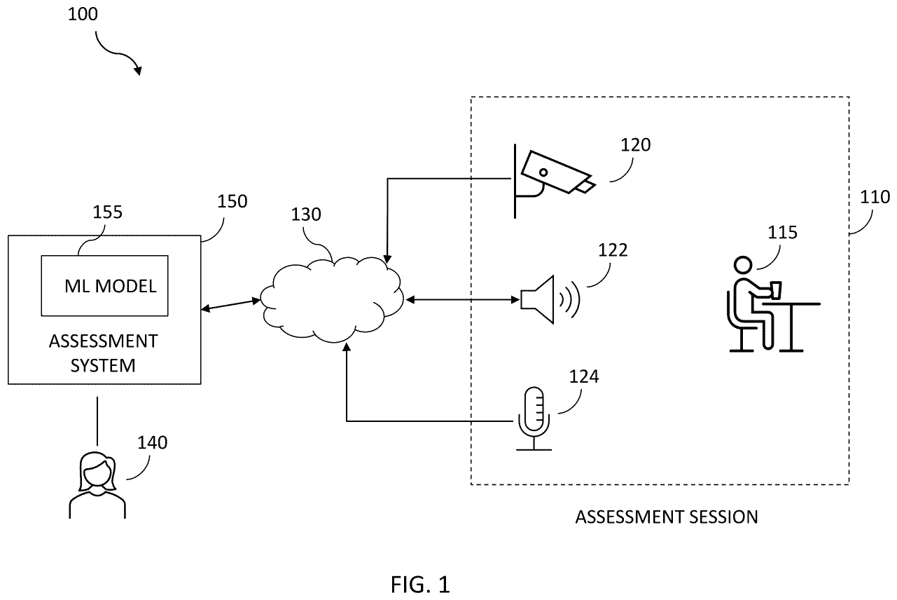

FIG. 1 is a block diagram illustrating a suitable network environment 100 for generating reports associated with an ASD assessment of a patient. An assessment session 110 includes a patient 115 (e.g., a child or other subject) that performs actions, behaviors, expressions, and so on, during the session 110. A camera 120 or other video capture component may capture a stream of video (e.g., one or more video clips or images) during the assessment. In some cases, the camera 120 may be a camera of a mobile device held by a parent or caregiver of the patient 115.

The patient 115 may hear audio cues or voice commands played by a speaker 122 or other audio component (e.g., a speaker of a parent's mobile device). In some cases, the voice commands may be spoken by an expert evaluator 140 located remotely (over a network 130) from the assessment session 110. During the session 110, the patient 115 hears voice commands or instructions (e.g., commands consistent with the Diagnostic and Statistical Manual of Mental Disorders (DSM-5), The Modified Checklist for Autism in Toddlers (M-CHAT-R) designed for toddlers between 16 and 30 months of age, the Autism Diagnostic Observation Schedule (ADOS-2), SRS-2, CARS, AGAS-2), and so on).

In response, the patient 115 performs actions or otherwise reacts to the voice commands. For example, the patient 115 may be instructed to “grab a few gold-fish crackers for a well-deserved snack” or a parent blows bubbles and the child is asked to “grab one of the bubbles.” The camera 120 may capture video of the patient 115 performing the actions (e.g., finding and eating the crackers) or otherwise reacting to the voice commands. Further, a microphone 124 or other audio capture device may capture audible responses uttered or spoken by the patient 115 and/or the audio cues or voice commands.

An assessment system 150 employs an ML model 155, receives audio data (e.g., the spoken commands and/or audible responses) and video data (e.g., a stream of video data captured during the assessment session 110) during or after the session 110. For example, the assessment system 140 may be associated with the expert evaluator 140 and located remotely from the assessment session 110.

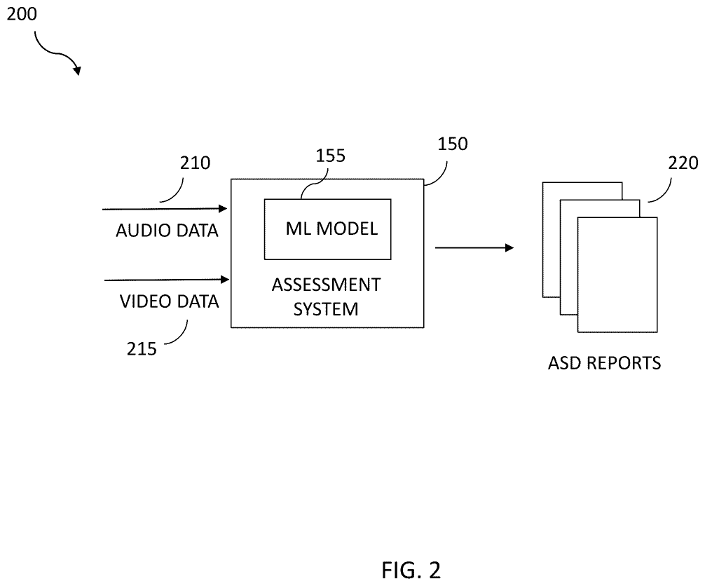

FIG. 2 is a block diagram illustrating various aspects of the assessment system 150. The assessment system 150 may include multiple modules implemented with a combination of software (e.g., executable instructions, or computer code) and hardware (e.g., at least a memory and processor). Accordingly, as used herein, in some embodiments, a module is a processor-implemented module and represents a computing device having a processor that is at least temporarily configured and/or programmed by executable instructions stored in memory to perform one or more of the particular functions that are described herein.

The assessment system 150 may include a multi-modal input processing module, which captures and processes audio and video feeds (e.g., audio data 210 and/or video data 215) from the assessment session 110 (e.g., a teleconferencing session). The multi-modal input processing module synchronizes the input feeds, aligning the voice commands with corresponding actions captured in the video feed (e.g., via pose estimation, temporal analysis, or other CV techniques).

The assessment system 150 also includes a voice command recognition module, which employs speech-to-text processing and natural language processing (NLP) techniques to interpret the voice commands directed towards the patient 115 and/or generate transcriptions of the voice commands and time-stamped audio data. For example, the voice command recognition module may determine intended actions or responses expected from the patient 115 based on the provided commands or audio cues (e.g., the audio data 210).

Further, the assessment system 150 also includes a computer vision module, which employs computer vision (CV) algorithms to analyze the video feed (e.g., the video data 215), extracting relevant visual cues and behaviors exhibited by the patient 115 during the assessment session 110. For example, the CV module may analyze facial expressions, gestures, posture estimation, motor skills, social interactions, and other behaviors, employing convolution neural networks (CNNs) and/or pose estimation models (e.g., part of ML model 155) to extract the visual cues and/or behaviors.

Also, the assessment system 150 includes a diagnostic inference module, which trains the ML model 155 based on machine learning and generative AI algorithms to analyze the synchronized audiovisual data and detect or determine patterns indicative of ASD. In some cases, the diagnostic inference module trains the ML model 155 using a diverse dataset of ASD assessments conducted by experts, to accurately identify potential symptoms and behavioral markers associated with ASD during the assessment session 110. As described herein, the ML model 155 may include or utilize transformers (BERT, GPT) for contextual analysis, and generate diagnostic probabilities, confidence scores, and/or structured reports.

The assessment system 150 may also include a report generation module, which integrates the outputs from the other modules to generate a comprehensive assessment report 220 or reports. The report 220 may include quantitative measures, qualitative observations, and/or recommendations for further evaluation or intervention.

In some cases, the assessment system 150 includes and/or is associated with a feedback interface (e.g., a Human-in-the-Loop), such as an intuitive interface for psychiatrists, psychologists, developmental pediatricians, clinicians, or healthcare professionals to review the assessment reports, annotate additional observations, and provide feedback to the ML model 155 for incremental improvement of various models or modules, and so on. Further, the assessment system 150 may implement various security, privacy, or compliance features, such as encryption, data anonymization, and other techniques that ensure patient privacy and security.

The assessment system 150 and the technology described herein may include and/or employ components, systems, servers, and devices that provide a general computing environment and network within which the technology described herein can be implemented. Further, the systems, methods, and techniques introduced here can be implemented as special-purpose hardware (for example, circuitry), as programmable circuitry appropriately programmed with software and/or firmware, or as a combination of special-purpose and programmable circuitry. Hence, implementations can include a machine-readable medium having stored thereon instructions which can be used to program a computer (or other electronic devices) to perform a process. The machine-readable medium can include, but is not limited to, floppy diskettes, optical discs, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other types of media/machine-readable medium suitable for storing electronic instructions.

The network 130 or cloud can be any network, ranging from a wired or wireless local area network (LAN) to a wired or wireless wide area network (WAN), to the Internet or some other public or private network, to a cellular network (e.g., 4G, LTE, 5G, 6G network), and so on. While the connections between the various devices and the network and are shown as separate connections, these connections can be any kind of local, wide area, wired, or wireless network, public or private.

Further, any or all components depicted in the Figures described herein can be supported and/or implemented via one or more computing systems or servers. Although not required, aspects of the various components or systems are described in the general context of computer-executable instructions, such as routines executed by a general-purpose computer, e.g., mobile device, a server computer, or personal computer. The system can be practiced with other communications, data processing, or computer system configurations, including: Internet appliances, hand-held devices, wearable devices, or mobile devices (e.g., smart phones, tablets, laptops, smart watches), all manner of cellular or mobile phones, multi-processor systems, microprocessor-based or programmable consumer electronics, set-top boxes, network PCs, mini-computers, mainframe computers, AR/VR devices, gaming devices, and the like. Indeed, the terms “computer,” “host,” and “host computer,” and “mobile device” and “handset” are generally used interchangeably herein and refer to any of the above devices and systems, as well as any data processor.

Aspects of the system can be embodied in a special purpose computing device or data processor that is specifically programmed, configured, or constructed to perform one or more of the computer-executable instructions explained in detail herein. Aspects of the system may also be practiced in distributed computing environments where tasks or modules are performed by remote processing devices, which are linked through a communications network, such as a Local Area Network (LAN), Wide Area Network (WAN), or the Internet. In a distributed computing environment, program modules may be located in both local and remote memory storage devices.

Aspects of the system may be stored or distributed on computer-readable media (e.g., physical and/or tangible non-transitory computer-readable storage media), including magnetically or optically readable computer discs, hard-wired or preprogrammed chips (e.g., EEPROM semiconductor chips), nanotechnology memory, or other data storage media. Indeed, computer implemented instructions, data structures, screen displays, and other data under aspects of the system may be distributed over the Internet or over other networks (including wireless networks), or they may be provided on any analog or digital network (packet switched, circuit switched, or other scheme). Portions of the system may reside on a server computer, while corresponding portions may reside on a client computer such as a mobile or portable device, and thus, while certain hardware platforms are described herein, aspects of the system are equally applicable to nodes on a network. In some cases, the mobile device or portable device may represent the server portion, while the server may represent the client portion.

### Examples of Generating ASD Reports

As described herein, in some embodiments, the assessment system 150 is configured to receive audio data (e.g., voice commands) synchronized (e.g., paired) to actions performed by a patient and captured in a video feed of the patient during an assessment and automatically generate diagnostic reports for the patient, such as reports that indicate or infer one or more likely ASD markers or impressions for the patient (or, indicate or infer one or more impressions that are contrary to ASD).

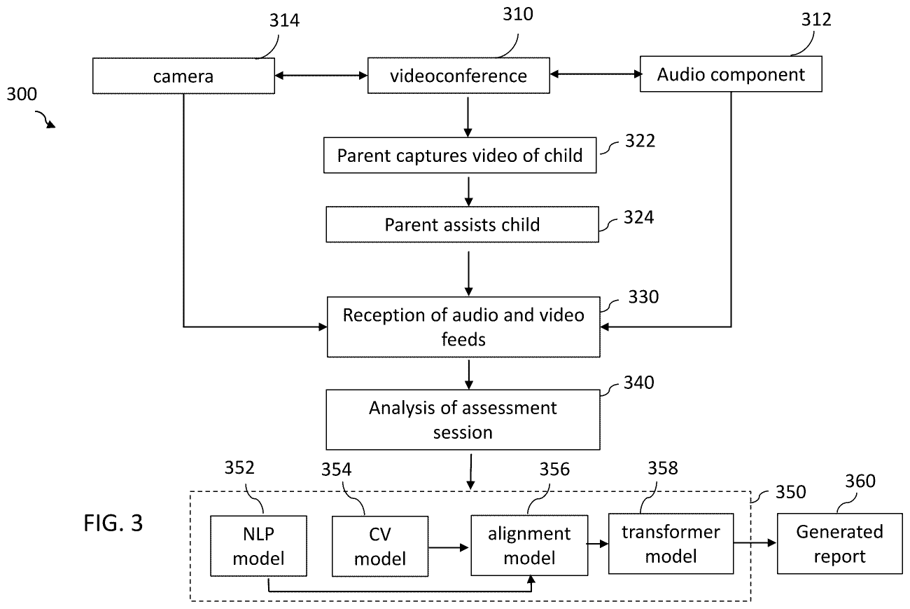

FIG. 3 is a flow diagram illustrating a method 300 of generating a diagnostic report for an assessment session, such as the session 110. Initially, a video conference 310 begins, where a connection between a video camera 314 observing a patient and an audio component 312 that present audio cues by an evaluator is established (see FIG. 1). As described herein, when the patient is a child, a parent or caregiver may capture the video 322 of the child and/or the parent (or another parent) assists 324 the child in perform actions and/or understanding the voice commands.

During or after the assessment session, the assessment system 150 receives audio feeds and video feeds 330 and performs an analysis 340 of the assessment session. The system 150 may employ various ML models 350 (e.g., the ML model 155) when analyzing the session.

An NLP model 352 performs voice command recognition and processes the audio feed to recognize and transcribe the voice commands issued by the expert evaluator. The NLP model 352 may utilize speech recognition techniques, such as automatic speech recognition (ASR) models or deep learning-based approaches, to convert the audio signals into text representations of the spoken commands. The transcribed voice commands may be timestamped to indicate exact or approximate moments in time when they were issued during the assessment session.

A CV model 354 performs action detection and analyzes the video feed to detect and identify the actions and responses of the patient during the assessment. The CV model 354 may utilize computer vision techniques, such as object detection, pose estimation, and activity recognition, to identify specific behaviors, gestures, and interactions exhibited by the patient. In some cases, the CV model 354 timestamps each detected action or response in the video feed, in order to align the action/response with a corresponding voice command issued by the expert evaluator and transcribed by the NLP model 352.

An alignment model 356 performs temporal alignment of the voice commands and the corresponding actions in the video feed (e.g., using the timestamps) to synchronize the two modalities. For example, the alignment model 365 matches each voice command with a closest corresponding action in the video feed based on their respective timestamps. The alignment model 356, in some cases, may attempt to minimize temporal discrepancies between the voice commands and the observed actions, ensuring or facilitating an accurate synchronization of voice commands to actions.

In some cases, the alignment model 356 may include error handling or correction mechanisms that mitigate potential inaccuracies or discrepancies that arise during the synchronization process. For example, the model 356 may implement techniques, such as dynamic time warping (DTW) or interpolation, to handle cases where the timestamps do not perfectly align due to noise, latency, or other factors. Further, the model 356 may incorporate feedback mechanisms that enable manual correction or adjustment of the synchronization, enabling human intervention to refine the alignment process. The alignment model 356, in some cases, generates paired data as an output, such as command-action pairs that are synchronized based on the timestamps.

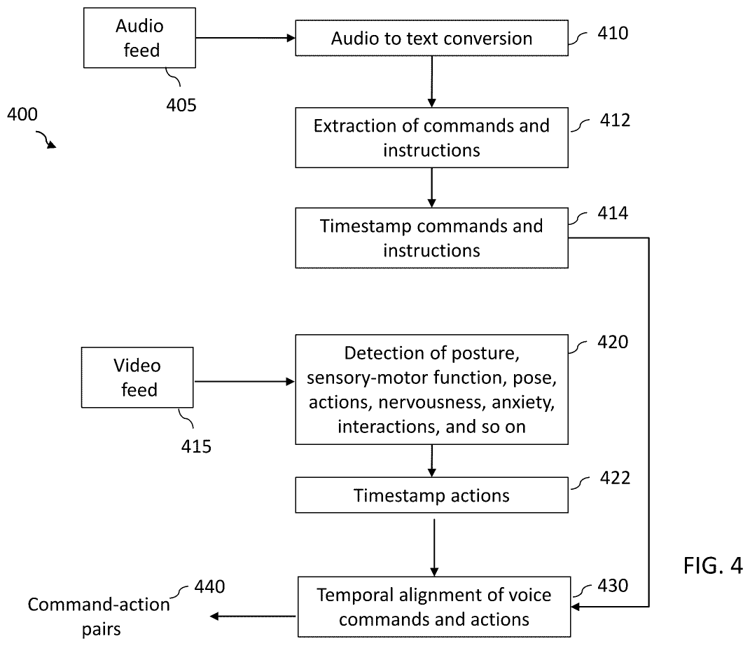

FIG. 4 is a flow diagram illustrating a method 400 of generating command-action pairs from audiovisual data of an assessment session. In step 410, the NLP model 352 performs audio to text conversion by applying NLP techniques to generate a text-based transcript of an audio feed 405. In step 412, the NLP model 352 extracts commands, instructions, cues, and so on, from the transcript, and in step 414, tags the extracted commands, instructions, cues, and so on, with timestamps and/or other metadata.

In step 420, the CV model 354 detects features or behaviors within a video feed 415. Example features or behaviors include postures, poses, actions or activities (e.g., actions that indicate nervousness, anxiety, and so on), interactions, movements, and so on. The CV model 354 may employ various CV techniques for feature detection, including pose estimation, keypoint detection, and so on. In step 422, the CV model 354 tags the extracted features or behaviors with timestamps and/or other metadata.

In step 430, the alignment model 356 receives the timestamped commands and the timestamped features/actions and performs a temporal alignment of the commands to the features/actions. The alignment model 365, in some cases, outputs one or more command-action pairs 440.

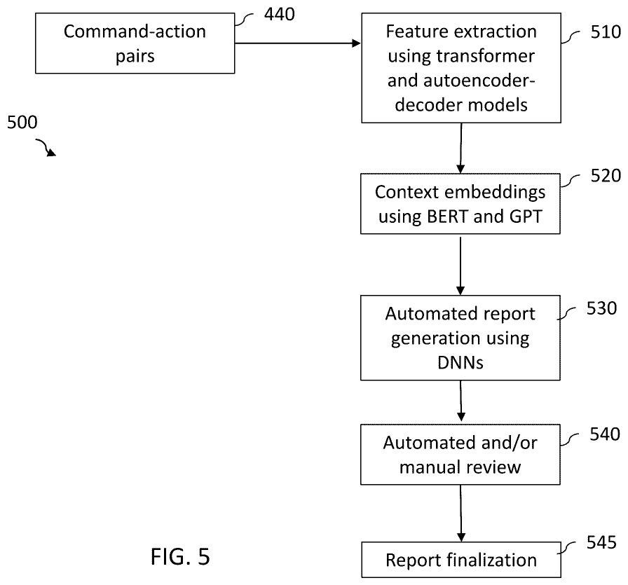

Returning back to FIG. 3, a transfer model 358 may receive the command-action pairs 440, or other similar output, and generate (automatically) a report 360 that includes inferences or other diagnostics for the patient. FIG. 5 is a flow diagram 500 illustrating the generation of a report using a transformer model (or another ML model).

In step 510, the transformer model 358 receives the aligned data (e.g., the command-action pairs 440) and performs feature extraction using various generative models or transformer-based architectures, such as transformer models, autoencoder-decoder models, and so on. In some cases, autoencoder-decoder models are employed to learn compact representations of the synchronized audiovisual data, capturing salient features and patterns present in the voice commands and the captured responses. In some cases, the transformer-based architectures process sequential data and capture long-range dependencies and are employed to analyze the temporal sequence of voice commands and corresponding actions observed in the video feed.

In step 520, the transformer model 358 employs or performs (using NLP) contextual analysis via transformer-based models, such as BERT (Bidirectional Encoder Representations from Transformers) or GPT (Generative Pre-trained Transformer). For example, using BERT, the model 358 may capture contextual embeddings of the transcribed voice commands, facilitating a nuanced understanding of semantic relationships and contextual cues present in the voice commands. As another example, using GPT, the model 358 generates contextualized representations of the synchronized data, facilitating the identification of key phrases, semantic cues, and contextual relationships between the voice commands and observed actions or features captured in the video data.

In step 530, the assessment system 150 performs diagnostic inference by applying ML models trained on a diverse dataset of ASD assessment reports. The system 150 may employ various deep neural networks, including CNNs, recurrent neural networks (RNNs), and so on. For example, the system 150 may employ CNNs to extract spatial features from the video feed and identify visual patterns and cues indicative of ASD-related behaviors and responses exhibited by the patient and/or employ RNNs to model temporal dependencies within the synchronized data and capturing sequential patterns and dynamics present in the voice commands and the actions/responses during the assessment session.

In step 540, the assessment system 150 performs an automated review and validation processes to ensure accuracy, coherence, and relevance of generated reports. The system 150 may utilize quality assurance mechanisms, such as by cross-referencing diagnostic inferences with established clinical guidelines, validating findings against previous assessment sessions, and/or detecting inconsistencies or discrepancies in the report contents. In some cases, the system 150, in step 545, may include or incorporate human oversight to validate the automated report and provide final approval before dissemination to clinicians, caregivers, or healthcare professionals.

In some embodiments, the technology utilizes continuous model learning, including some or all of the following aspects, when optimizing or enhancing the report generation techniques described herein.

For example, the system 150 may continuously accumulate new data from telehealth assessment sessions conducted over time. Example data includes synchronized audiovisual recordings, expert evaluator commands, child responses, annotated assessment reports, and so on. The system 150 may preprocess and/or augment the accumulated data to ensure consistency, quality, and diversity. For example, the system 150 may employ data cleaning, normalization, and/or augmentation techniques, such as data synthesis or data generation, to increase the diversity of the training dataset.

As another example, the system 150 may employ incremental model training techniques to continuously update and enhance the AI models used for automated report generation. New data batches may be periodically fed into the existing AI models, allowing the models to adapt and learn from the latest observations and insights gathered from telehealth assessment sessions. Incremental training may involve fine-tuning existing models using techniques such as transfer learning or online learning, where the model parameters are updated based on the new data without being retrained from scratch.

The system 150, after performing incremental training, may evaluate and validate the updated AI models to assess their performance and effectiveness. During an evaluation/validation, the system 150 may determine performance metrics, such as accuracy, precision, recall, and F1-score, to measure, for the model, the diagnostic accuracy and consistency with expert evaluations. In some cases, the system 150 may incorporate human validation to ensure that the updated models maintain high standards of quality and clinical relevance.

As another example, the system 150 may establish or utilize feedback mechanisms to incorporate insights and feedback from clinicians, domain experts, and/or caregivers into the model enhancement process. For example, clinicians and domain experts may review the generated assessment reports and provide feedback on the accuracy, relevance, and/or clinical utility of the diagnostic insights. Caregivers may also contribute feedback based on their observations and experiences during telehealth assessment sessions, providing useful input for model improvement.

Based on the received feedback, the system 150 may iteratively refine and improve its ML models over time. For example, the system 150 may adjust model parameters, architectures, and/or training strategies to address specific areas of improvement identified through feedback and performance evaluation. The iterative refinement process may ensure that the AI models continuously evolve and adapt to changing clinical needs, emerging diagnostic insights, and/or advancements in technology and healthcare practices.

As another example, the system 150 may develop explainable AI models to ensure transparency and interpretability of the diagnostic process. For example, the system 150 may employ transparency techniques, such as attention mechanisms, feature visualization, and/or model-agnostic interpretability methods, to provide insights into how the AI models arrive at their diagnostic decisions. Clinicians, caregivers, and other stakeholders may gain a deeper understanding of the factors influencing the diagnostic outcomes, fostering trust and confidence in the AI-generated assessment reports.

As another example, the system 150 may incorporate clinical interpretability features to facilitate meaningful interpretation of the diagnostic insights by healthcare professionals and clinicians. The system 150, via the generated reports, may present diagnostic findings in a structured and clinically relevant manner, with clear explanations of the observed behaviors, diagnostic criteria met, and/or recommendations for further evaluation and intervention. Thus, clinicians can easily interpret and contextualize the AI-generated assessment reports within a broader clinical context, enabling informed decision-making and personalized treatment planning for children with ASD, and other uses.

In some embodiments, the assessment system 150, as described herein, generates an ASD diagnostic report (with AI- or ML-generated content). The following information provides an example and/or generalized format for such reports (e.g., reports 220). Other reports may vary based on individual assessment findings, clinician preferences, evolving diagnostic practices, and so on.

Patient Information: The system 150 synthesizes demographic information about an individual, including age, gender, and/or relevant medical history, based on input data provided during the assessment session (e.g., via a patient intake survey). The system 150 utilizes input data from the telehealth assessment session, including audiovisual recordings, caregiver-provided information, conversational AI agents, and electronic health records, to populate this section or field of the report;

Assessment Details: The system 150 generates an overview of the assessment process, including the date of the assessment, names of evaluators, and summary of the assessment procedures conducted. The system 150 presents information about the assessment session, including timestamps of voice commands issued by the expert evaluator and synchronized with corresponding actions observed in the video feed;

Diagnostic Criteria: The system 150 summarizes the diagnostic criteria for ASD as outlined in established guidelines (such as the DSM-5), highlighting specific criteria met by the individual based on observed behaviors and assessment findings. The system 150 may present observations, social interactions, and communication patterns captured in the video feed, along with transcribed voice commands, that contribute to the identification of diagnostic criteria relevant to ASD;

Observations and Behaviors: The system 150 describes or introduces the observed behaviors, social interactions, repetitive behaviors, and communication patterns exhibited by the individual during the assessment session, providing specific examples and anecdotes. The system 150 may provide an analysis of audiovisual data, including facial expressions, gestures, and verbal responses, which informs the description of observed behaviors and interactions, ensuring a comprehensive assessment of the individual's behavior;

Diagnostic Impressions: The system 150, based on the observed behaviors and adherence to diagnostic criteria, provides diagnostic impressions, including a formal diagnosis of ASD or other neurodevelopmental disorders, along with a discussion of severity level and associated features. The system 150 may integrate information from all modalities, including behavioral observations, voice commands, and clinical data, to formulate diagnostic impressions consistent with established diagnostic guidelines and clinical expertise;

Recommendations: The system 150 may offer and/or present recommendations for further evaluation, intervention, and support services based on the diagnostic findings, including referrals to specialists, therapy services, and educational interventions. The system 150 may synthesize the recommendations based on the diagnostic impressions, aligning with the individual's specific needs and challenges identified during the assessment session;

Conclusion: The system 150 may summarizes the findings and recommendations presented in the diagnostic report, emphasizing the importance of early intervention, ongoing monitoring, and individualized support for individuals diagnosed with ASD. The system 150 may integrate information from all sections of the report to provide a coherent and comprehensive conclusion, highlighting the significance of the diagnostic process and the implications for the individual's care and well-being; and so on.

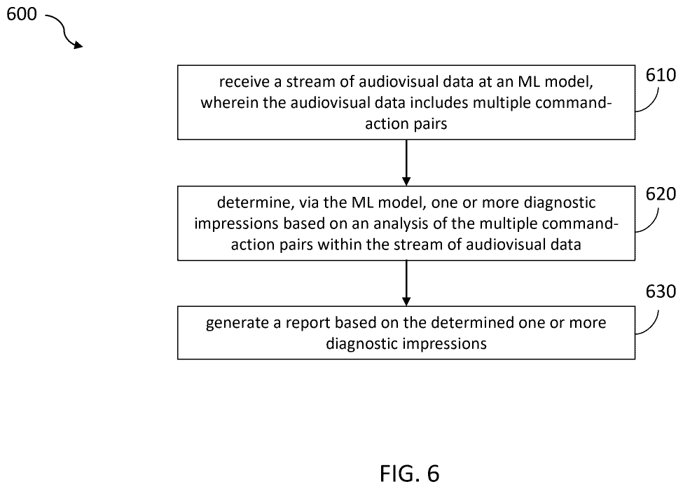

As described herein, the assessment system 150 may perform various methods or processes when automatically generating reports based on audiovisual data captured during an assessment session (or sessions) of a patient. FIG. 6 is a flow diagram illustrating a method 600 for generating a report associated with an assessment session. The method 600 may be performed by the assessment system 150 and, accordingly, is described herein merely by way of reference thereto. It will be appreciated that the method 600 may be performed on any suitable hardware.

In step 610, the assessment system 150 receives a stream of audiovisual data at an ML model, wherein the audiovisual data includes multiple command-action pairs. For example, each command-pair may include a text-based transcript of an audio command spoken to a subject and one or more images (e.g., video) of the subject responding to the audio command.

In step 620, the assessment system 150 determines, via the ML model, one or more diagnostic impressions based on an analysis of the multiple command-action pairs within the stream of audiovisual data. For example, the system 150 may receive the stream of audiovisual data via a multi-model input processing module of the ML model that synchronizes audio data to video data to align voice commands within the audio data to actions performed by a subject and captured within the video data, identify predicted actions or responses by the subject via a voice command recognition module that applies NLP to the audio data, extract visual features via a CV module that analyzes the video data of the subject, and detect one or more behavior patterns of the subject via a generative AI module that analyzes the extracted visual features synchronized to the identified predicted actions or responses by the subject.

In step 630, the assessment system 150 generates a report based on the determined one or more diagnostic impressions. For example, the report may include information identifying quantitative measures utilized during an assessment of the subject, information identifying qualitative observations associated with the determined one or more diagnostic impressions, and/or information identifying one or more recommendations based on the identified qualitative observations.

Thus, the assessment system 150, as described herein, may include an audio capture device that captures (or otherwise receives) audio cues spoken to the patient and/or audible patient responses during an assessment session, a video capture device that captures (or otherwise receives) a video feed of the patient during the assessment session, and a report generation component that automatically generates a report for the patient based on an analysis of the captured audio cues and the captured video feed (e.g., via one or more ML models).

The technology, therefore, facilitates remote ASD assessments, overcoming geographical barriers and increasing accessibility to specialized care, provides objective and standardized evaluation metrics, reducing reliance on subjective observations, enhances diagnostic accuracy and efficiency, leading to timely interventions and support for children with ASD, facilitates longitudinal monitoring and tracking of developmental progress over multiple assessment sessions, and other benefits.

### Example Embodiments of the Technology

The systems and methods described herein may be implemented as one or more embodiments, including:

A method, computer-readable medium, or system configured to receive a stream of audiovisual data by a machine learning (ML) model, wherein the audiovisual data includes multiple command-action pairs, determine, by the ML model, one or more diagnostic impressions based on an analysis of the multiple command-action pairs within the stream of audiovisual data, and generate a report based on the determined one or more diagnostic impressions.

In some cases, each command-action pair includes a text-based transcript of an audio command spoken to a subject and one or more images of the subject responding to the audio command.

In some cases, the ML model determines the one or more diagnostic impressions by receiving the stream of audiovisual data via a multi-model input processing module of the ML model that synchronizes audio data to video data to align voice commands within the audio data to actions performed by a subject and captured within the video data, identifying predicted actions or responses by the subject via a voice command recognition module that applies natural language processing (NLP) to the audio data, extracting visual features via a computer vision (CV) module that analyzes the video data of the subject, and detecting one or more behavior patterns of the subject via a generative artificial intelligence (AI) module that analyzes the extracted visual features synchronized to the identified predicted actions or responses by the subject.

In some cases, extracted visual features include facial expressions exhibited by the subject, gestures performed by the subject, or movements performed by the subject.

In some cases, the CV module analyzes the video data of the subject to extract the visual features by applying an object detection technique, a pose estimation technique, or an activity recognition technique.

In some cases, generating a report based on the determined one or more diagnostic impressions includes generating a report that includes information identifying quantitative measures utilized during an assessment of the subject, information identifying qualitative observations associated with the determined one or more diagnostic impressions, and information identifying one or more recommendations based on the identified qualitative observations.

In some cases, determining the one or more diagnostic impressions based on the analysis of the multiple command-action pairs within the stream of audiovisual data includes learning compact representations of the stream of audiovisual data via an autoencoder-decoder of the ML model, performing context analysis of the stream of audiovisual data via a transformer model, and performing diagnostic inference of the stream of audiovisual data via a deep neural network (DNN).

In some embodiments, a system for diagnosing autism spectrum disorder (ASD) in a patient includes an audio capture device that captures audio cues spoken to the patient and audible patient responses during an assessment session, a video capture device that captures a video feed of the patient during the assessment session, and a report generation component that automatically generates a report for the patient based on an analysis of the captured audio cues and the captured video feed.

In some cases, the report generation component includes a machine leaning (ML) model configured to generate the report, by receiving a stream of audiovisual data that synchronizes the captured audio cues to the captured video feed, determining one or more diagnostic impressions based on an analysis of the stream of audiovisual data, and generating the report based on the determined one or more diagnostic impressions.

In some cases, the stream of audiovisual data includes multiple command-action pairs; and wherein the one or more diagnostic impressions are determined based on an analysis of the multiple command-action pairs.

In some cases, a command-action pair is an audio cue mapped to an action performed by the patient during the assessment session in response to the audio cue.

In some cases, the report includes information identifying quantitative measures utilized during the assessment session, and information identifying qualitative observations based on the analysis of the captured audio cues and the captured video feed.

In some cases, the report generation component includes a multi-model input processing module that synchronizes the audio cues to the video feed to align voice commands within the audio cues to actions performed by the patient and captured within the video feed, a voice command recognition module that identifies predicted actions or responses by the patient by applying natural language processing (NLP) to the audio cues, a computer vision (CV) module that extracts visual features of the patient within the capture video feed, and a generative artificial intelligence (AI) module that detects one or more behavior patterns of the patient by analyzing the extracted visual features synchronized to the identified predicted actions or responses by the patient.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or”, in reference to a list of two or more items, covers all of the following interpretations of the word: any of the items in the list, all of the items in the list, and any combination of the items in the list.

The above detailed description of embodiments of the disclosure is not intended to be exhaustive or to limit the teachings to the precise form disclosed above. While specific embodiments of, and examples for, the disclosure are described above for illustrative purposes, various equivalent modifications are possible within the scope of the disclosure, as those skilled in the relevant art will recognize.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference. Aspects of the disclosure can be modified, if necessary, to employ the systems, functions, and concepts of the various references described above to provide yet further embodiments of the disclosure.

These and other changes can be made to the disclosure in light of the above Detailed Description. While the above description describes certain embodiments of the disclosure, and describes the best mode contemplated, no matter how detailed the above appears in text, the teachings can be practiced in many ways. Details of the technology may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features, or aspects of the disclosure with which that terminology is associated. In general, the terms used in the following claims should not be construed to limit the disclosure to the specific embodiments disclosed in the specification, unless the above Detailed Description section explicitly defines such terms. Accordingly, the actual scope of the disclosure encompasses not only the disclosed embodiments, but also all equivalent ways of practicing or implementing the disclosure under the claims.

From the foregoing, it will be appreciated that specific embodiments have been described herein for purposes of illustration, but that various modifications may be made without deviating from the spirit and scope of the embodiments. Accordingly, the embodiments are not limited except as by the appended claims.

What is claimed is:

1. A method, comprising:
receiving a stream of audiovisual data by a machine learning (ML) model, wherein the audiovisual data includes multiple command-action pairs;
determining, by the ML model, one or more diagnostic impressions based on an analysis of the multiple command-action pairs within the stream of audiovisual data; and
generating a report based on the determined one or more diagnostic impressions.

2. The method of claim 1, wherein each command-action pair includes:
a text-based transcript of an audio command spoken to a subject; and
one or more images of the subject responding to the audio command.

3. The method of claim 1, wherein the ML model determines the one or more diagnostic impressions by:
receiving the stream of audiovisual data via a multi-model input processing module of the ML model that synchronizes audio data to video data to align voice commands within the audio data to actions performed by a subject and captured within the video data;
identifying predicted actions or responses by the subject via a voice command recognition module that applies natural language processing (NLP) to the audio data;
extracting visual features via a computer vision (CV) module that analyzes the video data of the subject; and
detecting one or more behavior patterns of the subject via a generative artificial intelligence (AI) module that analyzes the extracted visual features synchronized to the identified predicted actions or responses by the subject.

4. The method of claim 3, wherein the extracted visual features include facial expressions exhibited by the subject, gestures performed by the subject, or movements performed by the subject.

5. The method of claim 4, wherein the CV module analyzes the video data of the subject to extract the visual features by applying an object detection technique, a pose estimation technique, or an activity recognition technique.

6. The method of claim 1, wherein generating a report based on the determined one or more diagnostic impressions includes generating a report that includes:
information identifying quantitative measures utilized during an assessment of the subject;
information identifying qualitative observations associated with the determined one or more diagnostic impressions; and
information identifying one or more recommendations based on the identified qualitative observations.

7. The method of claim 1, wherein determining the one or more diagnostic impressions based on the analysis of the multiple command-action pairs within the stream of audiovisual data includes:
learning compact representations of the stream of audiovisual data via an autoencoder-decoder of the ML model;
performing context analysis of the stream of audiovisual data via a transformer model; and
performing diagnostic inference of the stream of audiovisual data via a deep neural network (DNN).

8. A non-transitory computer-readable medium whose contents, when executed by a computing system, cause the computing system to perform a method, the method comprising:
receiving a stream of audiovisual data by a machine learning (ML) model, wherein the audiovisual data includes multiple command-action pairs;
determining, by the ML model, one or more diagnostic impressions based on an analysis of the multiple command-action pairs within the stream of audiovisual data; and
generating a report based on the determined one or more diagnostic impressions.

9. The computer-readable medium of claim 8, wherein each command-action pair includes:
a text-based transcript of an audio command spoken to a subject; and
one or more images of the subject responding to the audio command.

10. The computer-readable medium of claim 8, wherein the ML model determines the one or more diagnostic impressions by:
receiving the stream of audiovisual data via a multi-model input processing module of the ML model that synchronizes audio data to video data to align voice commands within the audio data to actions performed by a subject and captured within the video data;
identifying predicted actions or responses by the subject via a voice command recognition module that applies natural language processing (NLP) to the audio data;
extracting visual features via a computer vision (CV) module that analyzes the video data of the subject; and
detecting one or more behavior patterns of the subject via a generative artificial intelligence (AI) that analyzes the extracted visual features synchronized to the identified predicted actions or responses by the subject.

11. The computer-readable medium of claim 10, wherein the extracted visual features include facial expressions exhibited by the subject, gestures performed by the subject, or movements performed by the subject.

12. The computer-readable medium of claim 11, wherein the CV module analyzes the video data of the subject to extract the visual features by applying an object detection technique, a pose estimation technique, or an activity recognition technique.

13. The computer-readable medium of claim 8, wherein generating a report based on the determined one or more diagnostic impressions includes generating a report that includes:
information identifying quantitative measures utilized during an assessment of the subject;
information identifying qualitative observations associated with the determined one or more diagnostic impressions; and
information identifying one or more recommendations based on the identified qualitative observations.

14. The computer-readable medium of claim 8, wherein determining the one or more diagnostic impressions based on the analysis of the multiple command-action pairs within the stream of audiovisual data includes:
learning compact representations of the stream of audiovisual data via an autoencoder-decoder of the ML model;
performing context analysis of the stream of audiovisual data via a transformer model; and
performing diagnostic inference of the stream of audiovisual data via a deep neural network (DNN).

15. A system for diagnosing autism spectrum disorder (ASD) in a patient, the system comprising:
an audio capture device that captures audio cues spoken to the patient and audible patient responses during an assessment session;
a video capture device that captures a video feed of the patient during the assessment session; and
a report generation component that automatically generates a report for the patient based on an analysis of the captured audio cues and the captured video feed.

16. The system of claim 15, wherein the report generation component includes a machine leaning (ML) model configured to generate the report, by:
receiving a stream of audiovisual data that synchronizes the captured audio cues to the captured video feed;
determining one or more diagnostic impressions based on an analysis of the stream of audiovisual data; and
generating the report based on the determined one or more diagnostic impressions.

17. The system of claim 16, wherein the stream of audiovisual data includes multiple command-action pairs; and wherein the one or more diagnostic impressions are determined based on an analysis of the multiple command-action pairs.

18. The system of claim 17, wherein a command-action pair is an audio cue mapped to an action performed by the patient during the assessment session in response to the audio cue.

19. The system of claim 15, wherein the report includes:
information identifying quantitative measures utilized during the assessment session; and
information identifying qualitative observations based on the analysis of the captured audio cues and the captured video feed.

20. The system of claim 15, wherein the report generation component includes:
a multi-model input processing module that synchronizes the audio cues to the video feed to align voice commands within the audio cues to actions performed by the patient and captured within the video feed;
a voice command recognition module that identifies predicted actions or responses by the patient by applying natural language processing (NLP) to the audio cues;
a computer vision (CV) module that extracts visual features of the patient within the capture video feed; and
a generative artificial intelligence (AI) module that detects one or more behavior patterns of the patient by analyzing the extracted visual features synchronized to the identified predicted actions or responses by the patient.
